(19) United States
(12) Patent Application Publication
(10) Pub. No.: US 2020/0069250 A1
(43) Pub. Date: Mar. 5, 2020

(54) METHOD OF FORMING A THREE-DIMENSIONAL CONDUCTIVE KNIT PATCH

(71) Applicant: Myant Inc., Toronto (CA)

(72) Inventors: Tony Chahine, Toronto (CA); Gabriel Stefan, Toronto (CA)

(21) Appl. No.: 16/492,787

(22) PCT Filed: Mar. 12, 2019
(86) PCT No.: PCT/CA2018/000053

§ 371 Date: Sep. 10, 2019

Related U.S. Application Data
(60) Provisional application No. 62/469,581, filed on Mar. 10, 2017.

(51) Int. Cl. A61B 5/00; D04B 1/22

(52) U.S. Cl. CPC A61B 5/6804; D04B 1/22; A61B 2562/125; D10B 2401/16; D10B 2401/18

(57) ABSTRACT

A method of forming a three-dimensional conductive patch, the three-dimensional conductive patch forming a base layer coupled to one or more loop sections extending transverse o the base layer is disclosed. The method comprising forming the base fabric surface by interlacing a plurality of fibres including non-conductive fibres; forming a first segment including conductive fibres as a first portion of the three-dimensional conductive patch by interlacing a plurality of the conductive fibres transverse to the base fabric surface, the first portion interlaced with a first base fibre of the base fabric surface at one end and in a direction to an apex distanced from the base surface layer at another end of the first segment; and forming a second segment as a second portion of the three-dimensional conductive patch by interlacing a plurality of fibres including the conductive fibres extending in a direction from the apex to a second base fibre of the base surface layer; wherein the second portion is positioned relative to the first portion via the first base fibre and the second base fibre such that the first and second portions form a loop extending from the base fabric surface, the loop having the apex spaced apart from the base fabric surface, the first portion, the second portion and the base fabric surface integral with each other.

### CROSS-REFERENCE TO RELATED APPLICATIONS

This application claims priority to U.S. Provisional Patent Application No. 62/469,581, filed on Mar. 10, 2017; the entire contents of which are incorporated by reference herein.

### FIELD

The present disclosure relates to a conductive knit patch. More specifically, the present disclosure relates to a method of forming a three-dimensional conductive knit patch.

### BACKGROUND

A person's body emits signals which may be detected by appropriate electronic devices comprising one or more electrodes or other conductive patches that are positioned to be in contact with the person's skin. Generally, to maintain contact with the person's skin, the electrodes are glued to the skin or strapped in place. The electrodes are then connected by appropriate conductive leads to a monitoring device. This type of configuration can often be uncomfortable for the person and difficult to implement if the person is to remain clothed while the signals emitted by the body are monitored. Further, this configuration is not amenable for use when a person is moving, such as an athlete or a person walking.

Accordingly, electrically-conductive threads have been incorporated into garments for providing clothing with conductive patches forming sensors and electrical pathways to connect to monitoring devices for monitoring signals from a person's body. Previous solutions provide electrically-conductive threads forming conductive patches integrally knit or woven into a fabric layer, where the conductive patches are flush with the fabric layer. Accordingly, these garments with integrated conductive patches as sensors of the previous solutions do not maintain contact between the conductive patches as sensors and the person's body as the conductive patches forming the sensors move and shift as the fabric layer moves during wearing. Movement of the sensors inhibits accurate monitoring of the signals emitted by the [symbol] dy of the wearer as the sensors generally need to remain in contact with a specific location of the wearer's body to monitor the body's signals.

### BRIEF DESCRIPTION OF THE DRAWINGS

Embodiments of the present disclosure will now be described, by way of example only, with reference to the attached Figures.

FIG. 1A illustrates a perspective view of an example conductive knit patch.

FIG. 1B illustrates a zoomed-in view of a second example conductive knit patch that is being bent to expose the height/loft of the conductive fabric.

FIG. 2 illustrates a top down view of a single segment of an example conductive knit patch in an expanded form

FIG. 3A illustrates a top down view of a single segment of an example conductive knit patch in a looped form.

FIG. 3B illustrates a cross sectional view of a single segment of the example conductive knit patch of FIG. 3A in a looped form.

FIG. 3C illustrates a SANTONI® pattern for a conductive knit patch that is similar to FIG. 3A.

FIG. 4A illustrates a cross sectional view of a single segment of the example conductive knit patch of FIG. 4A in a looped form.

FIG. 4B illustrates a SANTONI® pattern for a conductive knit patch that is similar to FIG. 4B.

FIG. 5 illustrates a cross sectional view of a single segment of the example conductive knit patch of FIG. 5A in a looped form.

FIG. 6A illustrates a cross-sectional view of an example conductive knit patch having three segments segment that are all of equal height/loft.

FIG. 6B illustrates a SANTONI® pattern for the three segments segment of a conductive knit patch that is similar to FIG. 6A.

FIG. 6C illustrates a SANTONI® pattern for conductive knit patch having multiple segments segment as knit on a base fabric.

FIG. 7A illustrates a cross-sectional view of an example conductive knit patch having three segments (e.g. loops) where the edge segments have a lower height/loft than the central segment.

FIG. 7B illustrates a SANTONI® pattern for an example conductive knit patch having three segments where the edge segments have a lower height/loft than the central segment.

FIG. 8 illustrates a perspective view of an example conductive knit patch as integrally knit into a region of differing rigidity from the rest of the garment.

FIG. 9A illustrates a profile view of an example garment having an example conductive knit patch.

FIG. 9B illustrates a profile view of a second example garment having an example conductive knit patch.

FIG. 9C illustrates a profile view of a third example garment having an example conductive knit patch.

FIG. 9D illustrates a profile view of a fourth example garment having an example conductive knit patch.

FIG. 10 is an example of interlacing of the plurality of fibres of the layer of the garment.

FIG. 11 is a further embodiment of interlacing of the plurality of fibres of the layer of the garment.

### DETAILED DESCRIPTION

Disclosed herein is a method of forming a three-dimensional conductive knit patch. In one embodiment, the three-dimensional conductive knit patch can combine textiles, such as clothing, and microelectronics to form a wearable textile (e.g. a garment) having the knit patch.

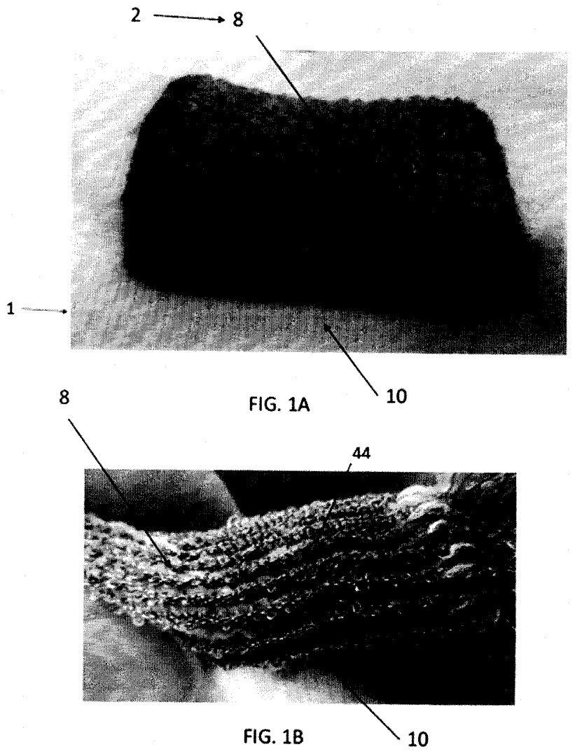

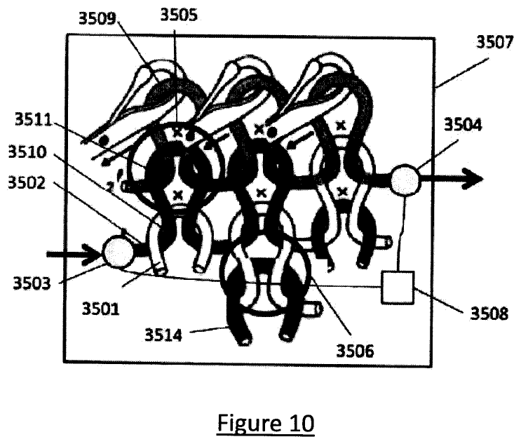

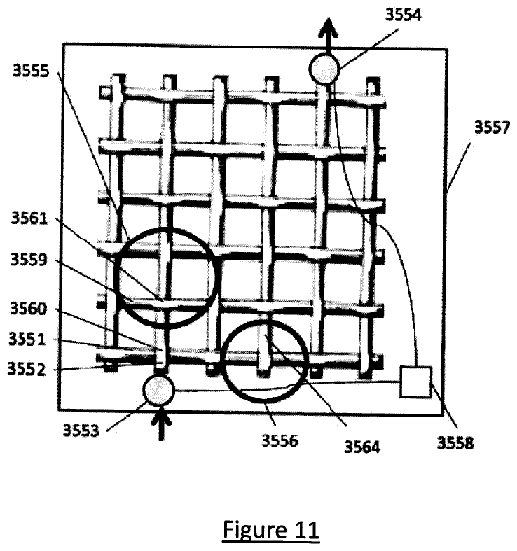

An example of a three-dimensional conductive knit patch 2 formable according to this disclosure is shown in FIG. 1A. In this example, three-dimensional conductive knit patch 2 consists of a base fabric (e.g. surface) 10 as a first portion integrally formed (e.g. knit) with a conductive fabric (e.g. group of conductive fibres) 8 as a second portion of a single layer 11 (see also FIG. 10). It is recognised that the fibres of the group of conductive fibres 8 (i.e. a patch) extend transverse to a surface layer of the base fabric 10.

It should be noted that herein, “integrated” or “integrally” refers to combining, coordinating or otherwise bringing together separate elements so as to provide a harmonious, consistent, interrelated whole. In the context of a textile, a textile can have various sections comprising networks of fibres with different structural properties. For example, a textile can have a section comprising a network of conductive fibres and a section comprising a network of non-conductive fibres. Two or more sections comprising networks of fibres are said to be “integrated” together into a textile (or “integrally formed”) when at least one fibre of one network is interlaced with at least one fibre of the other network such that the two networks form a layer of the textile. Further, when integrated, two sections of a textile can also be described as being substantially inseparable from the textile. Here, “substantially inseparable” refers to the notion that separation of the sections of the textile from each other results in disassembly or destruction of the textile itself. In manner of the patch 8, the fibre portions of the patch 8 extend from the base surface 10 as knit, i.e. a base fibre of the base surface 10 is used as a starting point to form a knit portion (i.e. combination of threads via knitting) extending transverse from the base surface 10, such that the knit fibres of the base surface 10 and the knit fibers of the fibre portion of the patch 8 share the same base fibre, i.e. the base fibre is knit into the base surface 10 as well as being [symbol] t into the knit fibre portion of the patch 8 extending transverse to the base surface 10.

In some examples, the conductive fabric (e.g. group of conductive fibres) 8 as a first portion can be knit transverse yet integral with the base fabric (e.g. surface) 10 layer 11, such as on but not limited to a SANTONI® circular knit machine. The base fabric surface 10 of the conductive knit patch 2 can be a part of a larger garment 1 such that the garment 1 incorporates the conductive knit patch 2. In some example embodiments, the conductive knit patch 2 can be integrally knit into a garment 1 on a SANTONI® circular knit machine. In other embodiments, the knit patch 2 can be knit or otherwise stitched/woven using other suitably configured interlacing machines.

Garment 1, e.g. a textile-based product, can be used by a user (such as a human, not shown). Garment 1 can include (but is not limited to) any one of a knitted textile, a woven textile, or a cut and sewn textile, a knitted fabric, a non-knitted fabric, a material that may or may not contact the user, a mat, a pad, a seat cover, etc., in any combination and/or permutation thereof (any equivalent thereof). The garment 1 can include an integrated functional textile article. It will be appreciated that some embodiments describe a knitted garment and it is understood that these embodiments may be extended to any textile fabric forms and/or techniques such as (weaving, knitting-warp, weft etc.), and the embodiments are not limited to a knitted garment. It will be appreciated that (where indicated) the Figures (drawings) may be directed to a knitted base fabric 10, and it will be appreciated that the base fabric 10 is an example of any form of textile fabrics and techniques such as (weaving, knitting - warp, weft etc.) for the base fabric 10, and that any description and/or illustration to the knitted garment fabric does this limit the scope of the present embodiments. In accordance with an embodiment, there is provided a garment 1 made with any textile forming technique (and the knitted fabric garment is simply an example of such an arrangement).

It should be noted that herein, “textile” refers to any material made or formed by manipulating natural or artificial fibres to interlace to create an organized network of [symbol] es. It is noted that the fibre portions extending transverse rom or extending transverse to the base surface 10 are considered in themselves as interlaced (e.g. knitted). Generally, textiles are formed using yarn, where yarn refers to a long continuous length of a plurality of fibres that have been interlocked (i.e. fitting into each other, as if twined together, or twisted together). Herein, the terms fibre and yarn are used interchangeably. Fibres or yarns can be manipulated to form a textile according to any method that provides an interlaced organized network of fibres, including but not limited to weaving, knitting, sew and cut, crocheting, knotting and felting. Exemplary structures (e.g. interlacing techniques) of textiles formed by knitting and weaving are provided in FIGS. 10 and 11, respectively. It should be noted that conductive fabric (e.g. group of conductive fibres) 8 can be formed as per the knitting structures as provided in FIG. 10. Conductive fabric (e.g. group of conductive fibres) 8 can also be formed as per the weaving structures as provided in FIG. 11. It should also be noted that base fabric surface 10 can be formed as per the knitting structures as provided in FIG. 10. Base fabric surface 10 can also be formed as per the weaving structures as provided in FIG. 11. Both portions 8 and 10 can be formed using the same interlacing technique. Further, portions 8 and 10 can be formed using the different interlacing techniques. Further, individual different loops 44 of portion 8 can be formed using different interlacing techniques.

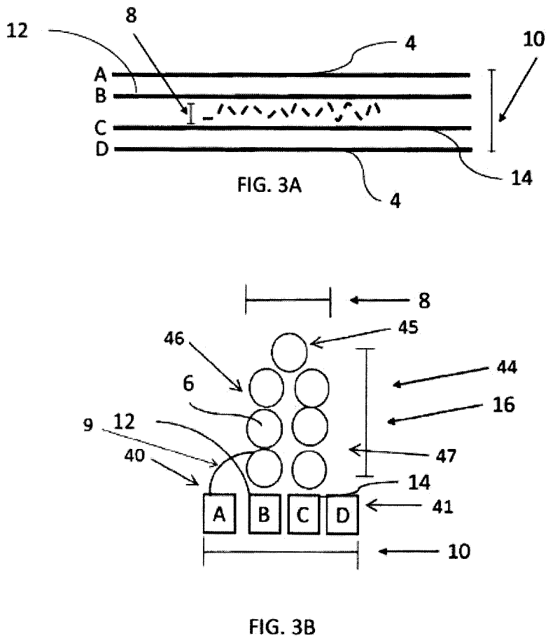

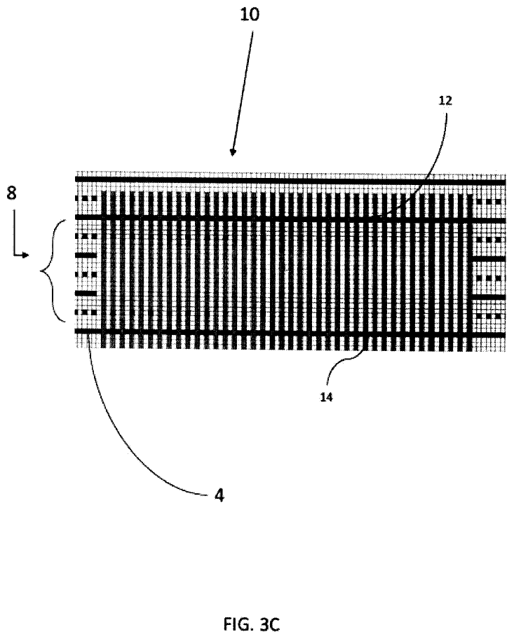

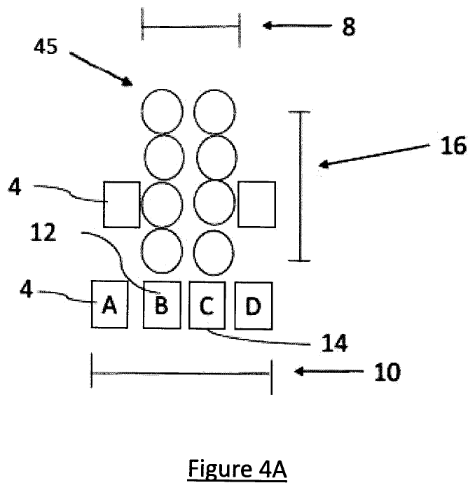

Different sections of a textile can be integrally formed into a layer to utilize different structural properties of different types of fibres. For example, conductive fibres can be manipulated to form networks of conductive fibres and non-conductive fibres can be manipulated to form networks of non-conductive fibers. These networks of fibres can comprise different sections of a textile by integrating the networks of fibres into a layer of the textile. Multiple layers of textile can also be stacked upon each other to provide a multi-layer textile. It is also recognized that the layer 11 can have the two portions 8, 10, such that portion 8 can extend from portion 10, i.e. when extending there is an angle 9 (see FIG. 3B) greater than 0 degrees and less than 180 degrees measured between the portions 8, 10 on either side of the intervening [symbol] se fibre 12, 14 (being the intersection point/location of the adjacent portions 8, 10), in which the portions 8, 10 extend in different directions from the base fibre 12, 14.

It should also be noted that herein, “interlace” refers to fibres (either artificial or natural) crossing over and/or under one another in an organized fashion, typically alternately over and under one another, in a layer. When interlaced, adjacent fibres touch each other at intersection points (e.g. points where one fibre crosses over or under another fibre). In one example, first fibres extending in a first direction can be interlaced with second fibres extending laterally or transverse to the fibres extending in the first connection. In another example, the second fibres can extend laterally at 90° from the first fibres when interlaced with the first fibres. Interlaced fibres extending in a sheet can be referred to as a network of fibres. Again, FIGS. 10 and 11, described below, provide exemplary embodiments of interlaced fibres.

As shown in FIGS. 1A and 1B, conductive fabric (e.g. group of conductive fibres) 8 can form a loop 44 (consisting of a plurality of fibres) having a height/loft 16 relative to the base fabric (e.g. surface) 10 of a garment 1 to such that the conductive knit patch 2 can contact a body of a wearer (e.g. user) of the garment 1 without the need for the base fabric surface 10 to contact the body of the wearer. This can be seen in FIG. 1B, which is a zoomed-in view of a three-dimensional conductive knit patch 2 shown as bent to expose individual components of the patch 2, including but not limited to conductive fabric 8 forming adjacent loops 44 and its corresponding height/loft 16. In this example, loops 44 of conductive fabric 8 of conductive knit patch 2 could contact the body of a wearer without base fabric 10 contacting the body of a wearer. A skilled person would understand that the height/loft 16 of loops 44 of the conductive knit patch 2 can independently vary based on how the conductive knit patch 2 is formed.

In some instances, contact of conductive knit patch 2 with a body part of a wearer can be enhanced (e.g. by incorporating conductive knit patch 2 into a compression garment (not shown), for example. A compression garment may press (e.g. compress) loops 44 of a conductive knit patch 2 having a height/loft 16 against body of a wearer. This can further enhance the contact of the conductive knit patch 2 against the body of the wearer.

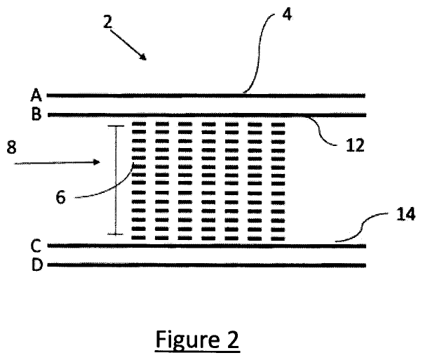

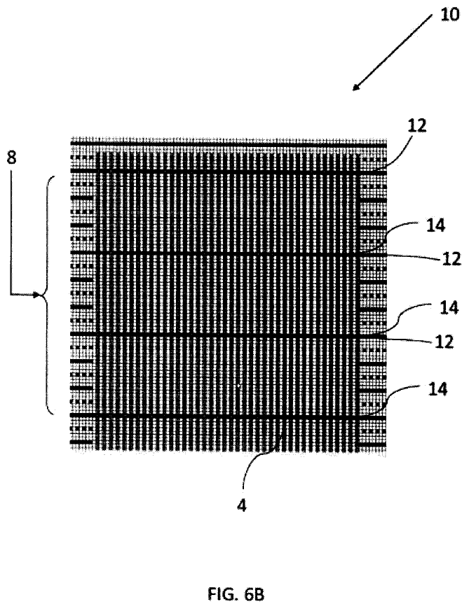

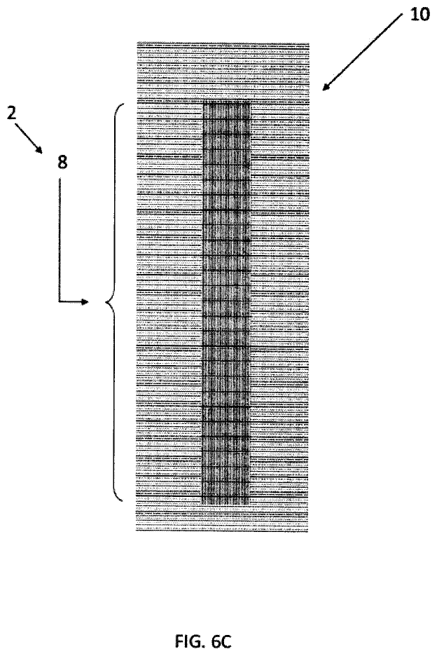

FIG. 2 is a top down view of a single segment (e.g. a single loop 44) of an example conductive knit patch 2. Specifically, FIG. 2 shows a plurality of non-conductive 4 and conductive 6 threads (e.g. fibres) extending from a first end 40 to a second end 41 of the base fabric surface 10. As shown in FIG. 6A by example, each loop 44 has two parts 46, 47 on either side of an apex 45 such that each part 46, 47 extends transversely from the base fabric surface 10 (i.e. the first portion 10). In particular, each part of the loop 44 is interlaced (e.g. knit) in a direction transverse to the base layer 10, such as in a transverse direction starting from base thread B towards the apex and then in a direction transverse to the base layer 10 from the apex back towards the base thread C.

FIG. 2 is provided to illustrate a top view of a conductive knit patch 2 may that be formed on a circular knit sewing machine, such as but not limited to a SANTONI® machine, providing a first configuration for forming of a conductive knit patch 2 according to the optional method described herein including incorporating first base yarn 12 and second base yarn 14.

In one example, the conductive knit patch 2 comprises a conductive fabric 8 (e.g. group of conductive fibres) as a second portion positioned between a first base yarn (e.g. fibre) 12 and a second base yarn (e.g. fibre) 14 within layer 11. Conductive fabric 8 can be made up of a plurality of conductive threads 6 interlaced together. Conductive fabric 8 can be interlaced with first base yarn (e.g. fibre) 12 and second base yarn (e.g. fibre) 14. In one example, the conductive fabric 8 can be interlaced (e.g. knit) with the first base yarn 12 at a first end 48 of the conductive fabric 8 and interlaced with the second base yarn 14 at a second end 49 of conductive fabric 8. It should be noted that conductive fabric 8 (e.g. group of conductive fibres) may comprise conductive fibres 6 as well as non-conductive fibres 4. It should be noted that first base yarn 12 can be second base yarn 14 and second base yarn 14 can be first base yarn 12.

Herein, non-conductive threads 4 may include, but are not limited to, synthetic fibers, natural fibers, and fibers derived from natural products. In certain embodiments, for instance, synthetic fibers may comprise (but are not limited to) nylon fibers, acrylic fibers, polyester fibers, and polypropylene fibers. In further embodiments, for example, yarns having a natural source may be obtained from cotton, wool, bamboo, hemp, alpaca and/or the like. In some embodiments, for instance, yarns derived from and/or manufactured from a natural source may be obtained from soy protein, corn, and the like. According to certain embodiments, for example, yarns having filament may have either a straight or textured form. Examples of such filament forms of yarn may include, but are not limited to, nylon, polyester, polypropylene and/or the like. The various yarns described herein, for instance, may be used individually or in combination with each other. Further, the yarn combinations may be formed, for example, in the knitting process or in a separate process prior to the knitting process. According to certain embodiments, for instance, the inlay yarn may include (but is not limited to) an elastomeric yarn comprising rubber, spandex or other elastic material such as Lycra® fiber. In further embodiments, for instance, the elastomeric yarns may further comprise a covering of straight and/or textured filament yarns such as nylon, polyester or polypropylene.

Conductive threads 6 may include X-STATIC® thread, metal-coated threads, or any thread that is configured to conduct electricity. For example, conductive threads 6 can be made of any conductive material including conductive metals such as stainless steel, silver, aluminium, copper, etc. In one embodiment, the conductive thread can be insulated. In another embodiment, the conductive thread can be uninsulated.

The following is an example of the steps of one method to form (e.g. knit) a three-dimensional conductive knit patch 2 with a single segment (e.g. loop 44). A skilled person would understand that a three-dimensional conductive knit patch 2 can also be formed with several segments (e.g. loops 44). Further, a skilled person would understand that the method of formation could be appropriate in situ three-dimensional stitching (e.g. weaving, knitting) techniques, such that one side of each loop of the [symbol] eductive knit patch 8 is knit in a line (e.g. a column extending from the base surface layer 10) extending transverse away from the base surface 10 to an apex of the loop and then in a second line (e.g. a second column extending from the apex towards the base surface layer 10) extending away from the apex of the loop towards the base surface 10, such that the second line is also in a direction transverse from the base surface 10. Each of the lines or columns of the sides of the loop 44 can consist of a series of rows extending from one side of the patch 8 to the other side of the patch, such that each side of the loop can be constructed in successive rows from side to side as the column is being knit in the direction of a line extending transverse to the base layer 10 (e.g. either from the first base fibre towards the apex or from the apex towards the second base fibre of the base surface layer 10. For example, the base surface layer 10 can be interlaced to one side of the patch 8, then the first base fibre common to both the base layer 10 and the first side of the patch 8 can be used to change direction of the interlacing such that the new direction for the first side of the loop 8 extends incorporates the first base fibre but at the same time begins to extend in the line direction transverse to the base layer 10. The interlacing continues until a series of interlaced rows (side to side) resulting in a column of multiple rows (or a series of columns interlaced from one side to the other side of the patch 8 resulting in a row of multiple columns) to form the first side of the loop extending from the base fibre to the apex. Once the apex is reached, the interlacing continues in a second line direction from the apex towards the soon to be second base fibre of the base layer 10 (such that the first and second base fibre layers are adjacent or otherwise proximal to one another in the base layer 10). As such, the interlacing continues in the second line direction (e.g. opposite to the first line direction) until a series of interlaced rows (side to side) resulting in a column of multiple rows (or a series of columns interlaced from one side to the other side of the patch 8 resulting in a row of multiple columns) forms the second side of the loop 44 extending from the apex to the second base fibre. At that point, the interlacing can once again change direction and either resume interlacing along the base layer surface 10 or to begin the next loop 44 of the patch 8 repeating the first side of the second loop 44 (adjacent to the first loop 44) to its apex and then back down to the next base fibre in the base surface layer 10, as noted [symbol] ove for forming of sides of the loops 44. Once the loops 44 of the patch 8 have been completed, the interlacing can continue along the original base surface layer direction 10 as desired to continue interlacing of the garment 1 itself incorporating the patch 8 and/or finish the edge of the patch 8, as desired.

In particular, it is noted that the direction of construction of the interlacing of the fibres of the first portion (i.e. along the first line) is opposite to the direction of construction of the interlacing of the fibres of the second portion (i.e. along the second line).

Circular knitting of the interlacing for the garment 1 and the patch 8 is defined as circular knitting or knitting in the round as a form of knitting that creates a seamless tube. When knitting circularly, the knitting is cast on and the circle of stitches is joined. Knitting is worked in rounds in a spiral. Originally, circular knitting was done using a set of four or five double-pointed needles. Later, circular needles were invented, which can also be used to knit in the round: the circular needle looks like two short knitting needles connected by a cable between them. Longer circular needles can be used to produce narrow tubes of knitting for the garment 1 and/or patch 8 (e.g. socks, mittens, and other items) using a Magic Loop technique. Machines also produce circular knitting; double bed machines can be set up to knit on the front bed in one direction then the back bed on the return, creating a knitted tube. Specialized knitting machines for garment 1 and/or patch 8 knitting use individual latch-hook needles to make each stitch in a round frame. Many types of garments 1 and or patches 8 can be knit in the round. Planned openings (e.g. patches 8) are temporarily knitted with extra stitches, reinforced if necessary. Then the extra stitches are cut to create the opening or allowance for the patch 8, and can be stitched with a sewing machine to prevent unraveling. This technique is called steeking. It is recognised that the apex of each loop 44 can be cut in order to separate each side of the loop 44 at the apex, as desired. It is also recognised that interlacing cab ne done to connect one base fibre to another adjacent or proximal base fibre as desired.

In the example embodiment shown in the Figures, base fabric surface 10 has non-conductive threads 4 labelled A, B, C, and D respectively. Non-conductive thread B is shown as a first base yarn 12 and non-conductive thread C is shown as a second base yarn 14, however, it should be noted that one or both of base yarns 12 and 14 can be conductive threads. Further, it should be noted that the position of first base yarn 12 and second base yarn 14 is not limited to the positions of conductive threads B and C, respectively, as shown in the Figures.

In one embodiment shown in FIG. 3A and FIG. 3B, base fabric surface 10 extends from a first side of the layer 11 to a second side of the layer 11 and from the first end 40 of the layer 11 to the second end 41 of the layer 11. FIG. 3A is a top down view of a single segment (e.g. loop) of an example three-dimensional conductive knit patch in a looped form. FIG. 3B is a cross sectional view of a single segment of the example conductive knit patch of FIG. 3A.

In one embodiment, a first segment 46 of base fabric surface 10 extends from first side of layer 11 to second side of layer 11 and from first end 40 of layer 11 to first base yarn 12. Second segment 47 of base fabric surface 10 extends from first side 42 of layer 11 to second side 43 of layer 11 and from second base fibre 14 to second base yarn 14.

In one example, conductive threads 6 can be positioned relative to (e.g. adjacent to) the first base yarn 12 (e.g. adjacent to first end 48 of the conductive fabric 8) and relative to the second base yarn 14 (e.g. adjacent to second end 49 of conductive fabric 8) in layer 11 such that the conductive threads 6 extend from fabric surface 10. For example, a conductive thread 6 can be interlaced (e.g. knitted) to an adjacent (e.g. neighboring) conductive thread 6 extending from fabric surface 10 (e.g. at first base fibre 12) to form a first portion of a second segment 44 (e.g. a loop 44). Subsequent conductive threads 6 can be interlaced to adjacent conductive threads 6 to form second segment 44 extending a distance 16 from first conductive fibre 12. In this manner, subsequent conductive threads 6 can be interlaced to adjacent [symbol] eductive threads 6 to form second segment 44 extending from first segment relative to first base fibre 12.

In one embodiment, a SANTONI sewing machine can be used to interlace conductive threads 6 extending from fabric surface 10 to apex 45 to form a first portion of the second segment 44 utilizing two needles. In one embodiment, one or more needles of the sewing machine are used to form the first portion 46 in a direction transverse to the first base fibre B. Subsequent conductive fibres 6 can be interlaced to each other as the first portion is built to increase distance 16 until apex 45 is reached.

Once first segment 46 of a desired conductive fabric 8 length is formed by interlacing a desired number of conductive fibres 6 extending from first base yarn 12 towards apex 45, a second portion 47 of second segment 44 of a desired conductive fabric length can be formed by interlacing a desired number of conductive fibres extending from apex 45 towards second base yarn 14. At second end of conductive fabric 8, conductive thread 6 can be interlaced with second base yarn 14. In another embodiment, conductive thread 6 positioned at second end of conductive fabric 8 can be coupled (e.g. knitted) to the second base yarn 14.

In one embodiment, a SANTONI knitting machine can be used to interlace conductive threads 6 extending from apex 45 to second base yarn 14 to form the second portion 47 of the second segment 44 by shifting the one or more needles of the knitting machine in a direction towards the base layer 10 and away from the apex 45. Subsequent conductive fibres 6 can be interlaced to each other to form the second portion 47 of second segment 44.

In one embodiment, upon positioning non-conductive threads 4 as a first segment relative to (e.g. adjacent to) first base yarn 12 within base surface 10, a first portion of the second segment can be formed by interlacing subsequent conductive or non-conductive fibres to first base yarn 12.

In another embodiment, a SANTONI® circular knit machine equipped with two needles can be used to form the various portions 46, 47 of the patch for each segment 44 of a multi-segment patch 8, each needle interlacing conductive or non-conductive fibres sequentially to form the first segment 46 from the base thread B to the apex 45. Upon completion of the first segment 46 to the apex 45, each needle interlacing conductive or non-conductive fibres sequentially is done to form the second segment 47 from the apex 45 to the base thread C adjacent to the base thread B. It is recognised that as part of completing the second segment 47, the base thread C could be interlaced with the adjacent base thread B in order to couple the base threads B, C to one another.

It should be noted that herein the term “adjacent” can generally refers to two components touching (e.g. in contact with each other) but is not limited to two components touching. For example, first base fibre 12 and the second base fibre 14 can be adjacent to each other such that first base fibre 12 and the second base fibre 14 are touching each other, however, first base fibre 12 and the second base fibre 14 being adjacent to each other can also refer to first base fibre 12 and the second base fibre 14 being in contact through an intermediary object such as but not limited to a piece of fabric or any other appropriate object. Intermediary object refers to an object that is touching (e.g. in contact with or adjacent to) both first base fibre 12 and the second base fibre 14, for example. In another embodiment, two objects being “adjacent” can refer to the two objects being interlaced with each other.

In one embodiment of the method of forming a conductive knit patch 2 described herein, first base yarn 12 and the second base yarn 14 are positioned relative to the second segment such that the second segment forms a bend or loop 44, the bend or loop 44 having a height/loft 16 relative to the base fabric 10. It should be noted that this is one optional method of forming loop 44 and that various in situ three-dimensional stitching (e.g. knitting) technologies can be used to form loop 44, i.e. one or more needles used to interlace the fibres of the portions 46, 47 in directions transverse to a base surface layer 10. In one embodiment, conductive knit patch 8 [symbol] ends from base fabric surface 10 by height/loft 16 of loop 44 towards a body of a user.

Regardless of the method of forming conductive knit patch 8, loop 44 can extend from base surface 10 such that loop 44 is adjacent to base fabric surface 10. In one embodiment, loop 44 extends from base fabric surface 10 in a direction transverse to base fabric surface 10.

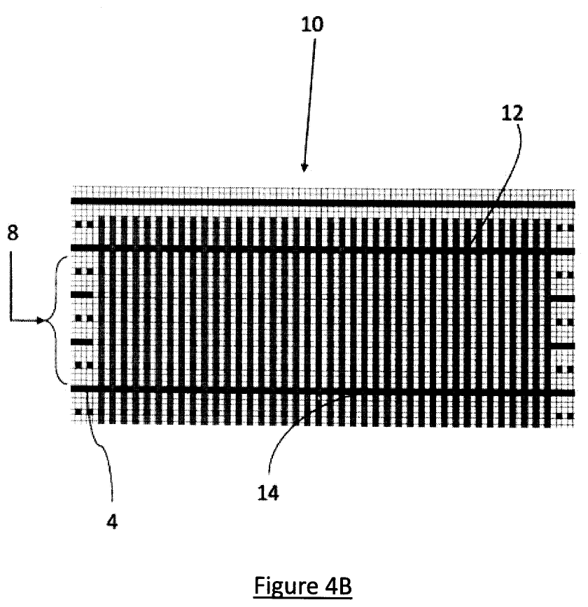

Loop 44 has an apex 45 of one or more fibres distal to (e.g. spaced apart from) base fabric surface 10. Apex 45 can be but is not limited to a single fibre of the group of conductive fibres 8 (see for example FIG. 4A), a portion of a single fibre of the group of conductive fibres 8, or more than one fibre of the group of conductive fibres 8 (see for example FIG. 4A).

Loop 44 has a first part 46 of the loop 44 and a second part 47 of the loop 44. In one embodiment, first part 46 of loop 44 extends from first conductive fibre 12 of base surface 10 a distance of loft/height 16 towards apex 45 and second part 47 of the loop 44 is opposed to first part 46 and extends from second base fibre 14 of base surface 10 a distance of loft/height 16 towards apex 45. In another embodiment, first part 46 of loop 44 extends from first end 48 of conductive fabric 8 a distance of loft/height 16 towards apex 45 and second part 47 of the loop 44 is opposed to first part 47 and extends from second end 49 of conductive fabric 8 a distance of loft/height 16 towards apex 45.

In one embodiment, first part 46 of loop 44 is connected to second part 47 of loop 44 at apex 45. In another embodiment, first part 46 of loop 44 is connected to second part 47 of loop 44 at or adjacent to the base fabric layer 10. In another embodiment, first part 46 of loop 44 is connected to second part 47 of loop 44 between apex 45 and base fabric layer 10. In another embodiment, first part 46 of loop 44 is connected to second part 47 of loop 44 at apex 45 and base fabric surface 10. In another embodiment, first part 46 of loop 44 and second part 47 of loop 44 are [symbol] arated (e.g. are not connected) from each other and form a furrow extending from first side of layer 11 to second side of layer 11.

In one embodiment, interlacing (e.g. knitting) conductive fabric 8 to be integral with fabric surface 10 within layer 11 can be repeated to form a conductive knit patch 2 with several segments (e.g. loops 44). For example, a second segment (e.g. loop 44) having its own conductive fabric (e.g. group of conductive fibres) 8 can be knitted to non-conductive thread D in order to knit a larger conductive knit patch 2, as shown in FIG. 6A and discussed hereafter.

In one optional example, once the single segment (e.g. loop 44) is formed, first base yarn 12 and second base yarn 14 can be connected to be integrated within layer 11. In other examples, first base yarn 12 and second base yarn 14 may be adjacent to each other prior to forming loop 44.

In another embodiment, first base yarn 12 and the second base yarn 14 can be stitched, knitted or woven together or otherwise connected by any appropriate manner known in the art. In another embodiment, first base yarn 12 and the second base yarn 14 can be connected by or fastened using any appropriate mechanical means such as but not limited to an adhesive (e.g. glue) or a hook-and-loop type fastener or by chemical modification.

In another embodiment, first base yarn 12 and the second base yarn 14 can be connected along a connecting line (not shown). In this embodiment, the connecting line can extend from first side 42 to second side 43 of layer 11 or can extend from second side 43 to first side 42. The connecting line can be straight or arcuate and can have any degree of curvature and/or number of bends. Further, the connecting line (not shown) can be a region of connection between first base yarn 12 and the second base yarn 14 that comprises more than one fibre (e.g. an area of fibres). In this embodiment, more than one fibre within either the base fabric surface 10 as the first portion or the group of conductive fibres 8 as the second portion can be connected [symbol] g. by any of the means previously described) to connect first base yarn 12 and the second base yarn 14 therewith.

As multiple conductive fabrics 8 are integrated into the layer 11 until the conductive knit patch 2 is of a suitable length for a desired application, conductive knit patch 2 can be manipulated to form a plurality of loops 44 (as described hereafter). For example, layer 11 may comprise a plurality of first base fibres 12 and second base fibres 14, each first base fibre 12 having a corresponding second base fibre 14 to form a pair of base fibres. By repeating the method of forming a conductive patch described above for each pair of base fibres, a conductive patch 2 can be formed comprising a plurality of adjacent and distinct loops 44, such that the respective parts 46, 47 are spaced apart from one another. For example, each part 46, 47 of loop 44 remains unconnected with an adjacent part 46, 47 of an adjacent loop once constructed between the base fibre 12, 14 and the apex 45 of each part 46, 47.

Further, it should be noted that second base fibre 14 can serve as a first base fibre 12 to an adjacent loop and first base fibre 12 can serve as a second base fibre 14 to an adjacent loop. It should also be noted that other methods of forming a three-dimensional conductive knit patch with a plurality of loops 44 could include, various in situ three-dimensional stitching (e.g. knitting) techniques (i.e. transverse to the base layer 10).

FIG. 3C roughly depicts a knit pattern diagram for the example conductive knit patch 2 of FIG. 3A-FIG. 3B for use in a SANTONI®-type circular knit machine. This example knit pattern shows the conductive fabric (e.g. group of conductive fibres) 8 (as shown by the gray pixel) being coupled to the first base yarn 12 (e.g. at a first end 48 of the conductive fabric 8) and coupled to second base yarn 14 (e.g. at a second end 49 of the conductive fabric 8). Note that non-conductive thread 4 is represented by a black pixel in FIG. 3C and white pixel represents either a no-knit or a drop stitch.

In another example, the conductive fabric (e.g. group of conductive fibres) 8 includes one or more non-conductive threads 4, as shown in FIG. 4A and FIG. 4B. FIG. 4A is a cross sectional view of a single segment of the example conductive knit patch in a looped form.

In this example, non-conductive threads 4 can be interlaced (e.g. knitted) to one or more of the conductive threads 6 forming loop 44. These non-conductive threads 4 can be used to change the characteristics of the conductive fabric (e.g. group of conductive fibres) 8. For instance, non-conductive thread 4 connected to a side of parts 46, 47 (e.g. between base fibres 12, 14 and apex 45) can be used as additional support (i.e. to inhibit height/loft 16 from decreasing/compressing and/or to maintain parts 46, 47 as having height/loft 16) for the conductive threads 6 forming the conductive fabric (e.g. group of conductive fibres) 8, allowing for a longer conductive fabric (e.g. group of conductive fibres) 8. This longer conductive fabric (e.g. group of conductive fibres) 8 could then be used to form a higher (e.g. loftier) height 16 of loop 44 once the first base yarn 12 and the second base yarn 14 are brought together (e.g. gathered). It should be noted that non-conductive threads 4 attached to a side of parts 46, 47 (between the base thread 12, 14 and apex 45) can be connected to one another (Le. one thread 4 of one loop 44 can be connected to another thread 4 on an adjacent loop 44).

The non-conductive threads 4 can also be used to change other characteristics of the conductive knit patch 8. These characteristics include, but are not limited to, the elasticity, stretchability, rigidity, and/or density of the conductive knit patch 8.

FIG. 4B roughly depicts a knit pattern diagram for the example conductive knit patch similar to FIG. 4A for use in a SANTONI®-type circular knit machine. Note that non-conductive thread 4 is represented by a black pixel and conductive thread 4 is represented by a blue/gray pixel.

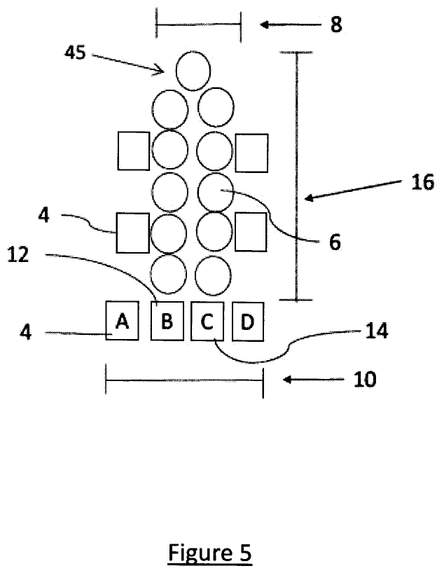

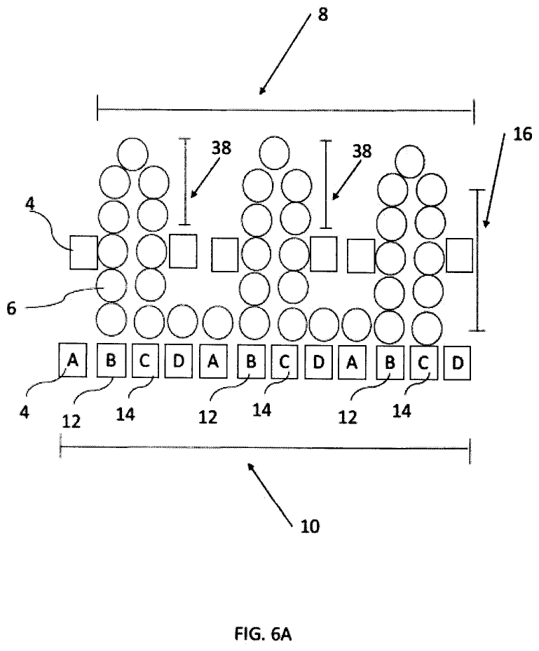

FIG. 5 depicts another example of a contact patch 2 having one or more non-conductive threads 4 in the conductive fabric (e.g. group of conductive fibres) 8 (similar to FIG. 4A-FIG. 4B). In this example, the additional non-conductive threads 4 allow for a longer conductive fabric (e.g. group of conductive fibres) 8 to be knit, allowing for a higher height/loft 16.

It should be understood that, depending on how the conductive knit patch 2 will be used, the method of forming three-dimensional conductive knit patch 2 described above can be performed repeatedly to create a conductive knit patch 2 of varying sizes (e.g. multiple loops 44 with varying height/lofts 16). In one example, a conductive knit patch 2 having a plurality of loops 44 is shown in FIG. 6A. In the example shown in FIG. 6A, the conductive knit patch 2 has a uniform height/loft 16. It should also be noted that in the example shown in FIG. 6A conductive thread 6 is knitted such that the conductive fabric (e.g. group of conductive fibres) 8 in each of the plurality of loops 44 is electrically connected. In this example, a conductive thread 6 is also interlaced (e.g. knit) to non-conductive threads D and A, adjacent to first end 48 of conductive fabric 8 and second end 49 of conductive fabric 49, respectively, so that the loops 44 of each segment of the conductive fabric (e.g. group of conductive fibres) 8 are electrically connected. In the example shown in FIG. 6, a conductive thread 6 is shown to be interlaced (e.g. knitted) to a non-conductive thread 4 adjacent to first end 48 of conductive fabric 8 and second end 49 of conductive fabric 49 such that conductive thread 6 is adjacent to base surface 10 within layer 11. Positioning conductive threads 6 adjacent to base surface 10 can provide for each segment (e.g. loop 44) of the conductive knit patch 2 to be electrically continuous (e.g. electrically connected).

In the example shown in FIG. 6A the loop area 38 contains only conductive thread 6 and does not contain any non-conductive thread 4. This example configuration may be useful in applications where only the conductive thread 4 should be in contact with the body. A skilled person, however, would understand that the configuration of nonconductive thread 4 and conductive thread 6 can vary depending on the application.

FIG. 6B roughly depicts a knit pattern diagram for the example conductive knit patch of FIG. 6A for use in a SANTONI®-type circular knit machine. This example knit pattern shows the conductive fabric (e.g. group of conductive fibres) 8 (as shown by the gray pixel) being connected to the first base yarn 12 and second base yarn 14. Note that non-conductive thread 4 is represented by a black pixel. Note that in this example, the second base yarn 14 can act as the first base yarn 12 for the subsequent segment. Other embodiments may separate the segments using one or more non-conductive threads 4.

FIG. 6C roughly illustrates a SANTONI® pattern for an entire conductive knit patch having multiple segments as knit on a base fabric 10. This knit pattern diagram shows the beginning and end edges of the conductive knit patch 8 as well as the multiple segments between the beginning and end edges of the conductive knit patch 8.

FIG. 6D illustrates a SANTONI® pattern for two entire conductive knit patches having multiple segments as knit on a base fabric 10. In this case, two conductive knit patches 8 would be knit side-by-side on a base fabric 10.

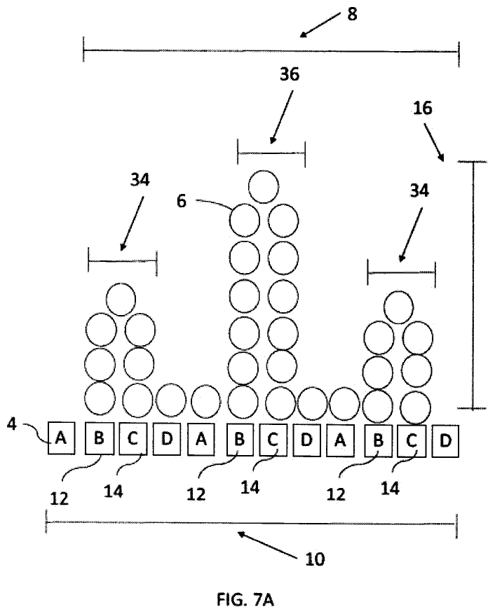

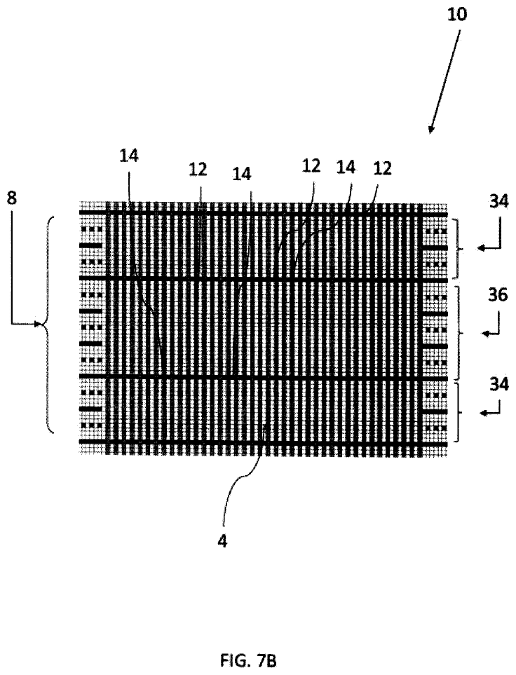

In another example, the conductive knit patch 8 can have areas with varying heights/lofts 16. An example of this is provided in FIG. 7A. FIG. 7A is a cross-sectional view of an example conductive knit patch 8 having a plurality of segments (e.g. loops 44) where the edge segments (e.g. loops) 34 have a lower height/loft 16 than the central segment (e.g. loop) 36.

In this example, unlike FIG. 6A, the height/loft 16 of the conductive knit patch 8 is higher at the center segment 36 than at the edge segments 34. In this example, the edge 10 segments 34 represent the edge of the conductive knit patch. In this case, the height differences between the edge segments 34 and the center segment 36 form a beveled edge which reduces the sideways/lateral spread of conductive knit patch 8. This can be useful in applications where many separate contact patches 8 are used in close proximity to each other. By reducing the [symbol] eways/lateral spread of a single conductive knit patch 2, adjacent loops 44 of conductive knit patches 8 are less likely to come in contact with one another. It should be clear that the contact of two adjacent conductive knit patches 8 may lead to unintentional electrical shorts when the conductive knit patches 8 are used in an electrical circuit.

Furthermore, similar to the example shown in FIG. 6A, the conductive thread 6 in FIG. 7A is knitted so that the conductive fabric (e.g. group of conductive fibres) 8 in each of the plurality of loops 44 is electrically connected. In this example, a conductive thread 6 is also knit to non-conductive threads D and A so that the loops of each segment of the conductive fabric (e.g. group of conductive fibres) 8 are connected. This allows for each segment of the conductive knit patch 8 to be electrically continuous.

FIG. 7B illustrates a SANTONI® pattern for an example conductive knit patch 8 having a plurality of segments where the edge segments 34 have a lower height/loft than the center segment 36. In this example it is evident that the length of the center segment 36 is longer than the edge segments 34. Once looped, this will result in the center segment 36 having a greater height/loft 16 than the edge segments 34. Note that in this example, the second base yarn 14 can act as the first base yarn 12 for the subsequent segment. Other embodiments may separate the segments using one or more non-conductive threads 4.

Further to the aforementioned embodiments, a conductive knit patch 8 can also be interlaced (e.g. knit) into a region (e.g. first region 30) of a garment 1 that has different fabric characteristics from other regions (e.g. second region 32) of the garment 1 such that movement of the conductive knit patch 8 with respect to an underlying part of a body can be altered and/or restricted (e.g. inhibited). Restricting (e.g. inhibiting) movement of conductive patch 8 with respect to the underlying body art of the wearer can promote the conductive knit patch 8 to maintain contact with the underlying part of the body of the user/wearer when the garment 1 is worn by a wearer.

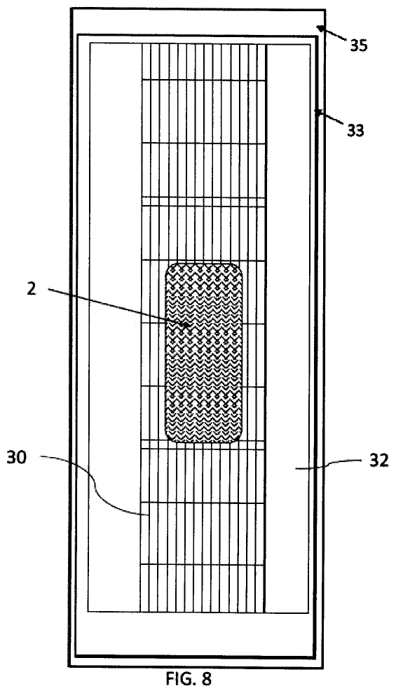

For example, FIG. 8 is a top down view of an example conductive knit patch 2 as integrally knit into a first region 30 having different fabric characteristics from the rest of the garment 1. In this example the conductive knit patch 2 is integrally knit into first region 30 of the garment 1 that has different fabric characteristics than its surrounding second region 32. These characteristics can include, but are not limited to, flexibility, elasticity, breathability, density, insulation, support, and compressibility. Ways of knitting regions of different fabric characteristics are known and can include but are not limited to, making the fabric knit denser relative to other parts of the garment; plastic or wire supports; iron-on, epoxy, resin, or adhesive fabric modifiers; and/or chemically treating the fabric.

In one example, garment 1 is such that the layer 11 can include a first region 30 containing one or more sensors (e.g. conductive patch 2) and a second region 32 adjacent to the first region 30, the first region 30 having a lower (e.g. less stretch or flexibility) degree of elasticity reflected by the plurality of fibres therein relative to a degree of elasticity reflected by the plurality of fibres in the second region 32; wherein the second region 32 contains non-conductive fibres for electrically insulating the one or more sensors from another conductive region (not shown) in the layer 11. It should be noted that the degree of elasticity reflected by the plurality of fibres in the second region 32 can vary across the second region 32. For example, a first section 33 of the second region 32 adjacent to the first region 30 can have a lower (e.g. less stretch or flexibility) degree of elasticity reflected by the plurality of fibres therein relative to a degree of elasticity in a second section 35 of the second region 32 distal (e.g. spaced apart from) to the first region 30. In this regard, second region 32 can have a plurality of sections, each section with a lower (e.g. less stretch or flexibility) degree of elasticity reflected by the plurality of fibres therein relative to a degree of elasticity in an adjacent region to create a gradient of elasticity across the plurality of section of the second region.

Further, the garment 1 can further comprise a plurality of fibres in the first region 30 that provide a thickness of the layer 11 greater than a thickness of the plurality of fibres in the second region 32.

Further, the garment 1 can further comprise a knit type of the plurality of the fibres in the first region 30 that is different from a knit type of the plurality of fibres in the second region 32, such that said difference is a factor providing the first region 30 having the lower degree of elasticity reflected by the plurality of fibres therein relative to the degree of elasticity reflected by the plurality of fibres in the second region 32. It should be noted as well that the each of the plurality of sections within region 32 can also comprise a knit type that is different from a knit type of an adjacent section of region 32 such that said difference is a factor providing the each section of the plurality of sections of second region 33 having the lower degree of elasticity reflected by the plurality of fibres therein, for example, relative to the degree of elasticity reflected by adjacent sections within the second region 32. The garment 1 is such that the plurality of the fibres in the first region 30 can include both the plurality of conductive fibres and non-conductive fibres, meaning sensor includes both conductive and non-conductive fibres.

Further, the garment 1 is such that the plurality of the fibres in the first region 30 can have a higher thread (e.g. knit) density (i.e. threads per inch) than the plurality of fibres in the second region 32, reflecting that the fibres of sensor 2 in the first region 30 are included in the higher thread density. Also, the garment 1 can be such that the plurality of the fibres themselves in the first region 30 can have a lower degree of elasticity than the plurality of fibres in the second region 32.

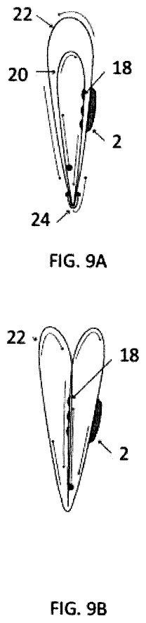

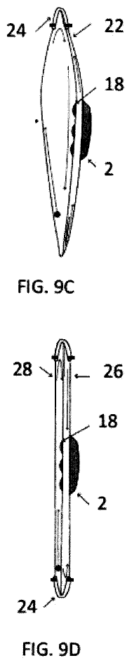

FIG. 9A-FIG. 9D are cross-sectional views of garments having an example conductive knit patch 2. In these example garments the conductive knit patch 2 is connected to a data bus 18 for conveying data. The data bus 18 may be connected to any kind of device used in an electrical system including, but not limited to, data processors, power supplies, actuators, sensors, and LEDS. In some examples the data bus is enclosed in an inner layer 20. In other example embodiments the data bus 18 may be on the inside of the fabric 26. In some other embodiments the data bus 18 may be exposed. In the examples provided in FIG. 9A-FIG. 9D, the conductive knit patch 2 and data bus 18 are part of a band-type garment such as a headband, wristband, or legband. In this example, the conductive knit patch 2 could [symbol] tact the body once the band-type garment is worn through the height/loft 16 of the conductive knit patch 2. In some other example embodiments, the height/loft 16 of the conductive knit patch 2 and the compression properties of the garment may be used to maintain contact with the body. In other embodiments the conductive knit patch 2 may be used to send and/or receive electrical signals, and/or to sense data from the body. Examples of sent signals include, but are not limited to, electrical muscle stimulation, or transcutaneous electrical nerve stimulation signals. Data sensed from the body can include, but is not limited to, moisture, conductivity, heart rate, etc.

In the embodiments described above, knitting can be used to integrate different sections of a garment 1 into layer 11. Knitting comprises creating multiple loops of fibre or yarn, called stitches, in a line or tube. In this manner, the fibre or yarn in knitted fabrics follows a meandering path (e.g. a course), forming loops above and below the mean path of the yarn. These meandering loops can be easily stretched in different directions. Consecutive rows of loops can be attached using interlocking loops of fibre or yarn. As each row progresses, a newly created loop of fibre or yarn is pulled through one or more loops of fibre or yarn from a prior row of the layer 11.

It should be noted that weaving can also be used to integrate different sections of garment 1 into a layer 11. Weaving is a method of forming a garment 10 in which two distinct sets of yarns or fibres are interlaced at a specified (e.g. right) angles to form the layer 11 of the garment 1.

FIG. 10 shows an exemplary knitted configuration of a network of electrically conductive fibres 3505 in, for example, a segment of an electric component (e.g. sensor 2). In this embodiment, an electric signal (e.g. current) is transmitted to conductive fibre 3502 from a power source (not shown) through a first connector 3503, as controlled by a controller 3508. The electric signal is transmitted along the electric pathway along conductive fibre 3502 past non-conductive fibre 3501 at junction point 3510. The electric signal is not propagated into non-conductive fibre 3501 at junction point 3510 because non-conductive fibre 3501 cannot conduct electricity. Junction point 3510 can refer to any point where adjacent conductive fibres and non-conductive [symbol] es are contacting each other (e.g. touching). In the embodiment shown in FIG. 10, non-conductive fibre 3501 and conductive fibre 3502 are shown as being interlaced by being knitted together. Knitting is only one exemplary embodiment of interlacing adjacent conductive and non-conductive fibres.

It should be noted that non-conductive fibres forming non-conductive network 3506 can also be interlaced (e.g. by knitting, etc.). Non-conductive network 3506 can comprise non-conductive fibres (e.g. 3501) and conductive fibres (e.g. 3514) where the conductive fibre 3514 is electrically connected to conductive fibres transmitting the electric signal (e.g. 3502).

In the embodiment shown in FIG. 10, the electric signal continues to be transmitted from junction point 3510 along conductive fibre 3502 until it reaches connection point 3511. Here, the electric signal propagates laterally (e.g. transverse) from conductive fibre 3502 into conductive fibre 3509 because conductive fibre 3509 can conduct electricity. Connection point 3511 can refer to any point where adjacent conductive fibres (e.g. 3502 and 3509) are contacting each other (e.g. touching). In the embodiment shown in FIG. 10, conductive fibre 3502 and conductive fibre 3509 are shown as being interlaced by being knitted together. Again, knitting is only one exemplary embodiment of interlacing adjacent conductive fibres.

The electric signal continues to be transmitted from connection point 3511 along the electric pathway to connector 3504. At least one fibre of network 3505 is attached to connector 3504 to transmit the electric signal from the electric component (e.g. sensor 2) to connector 3504. Connector 3504 is connected to a power source (not shown) to complete the electric circuit.

FIG. 11 shows an exemplary woven configuration of a network of electrically conductive fibres 3555. In this embodiment, an electric signal (e.g. current) is transmitted to conductive fibre 3552 from a power source (not shown) through a first connector 3553, as controlled by a controller 3558. The electric signal is transmitted along the electric component (e.g. sensor 2) along conductive fibre 3552 past non- [symbol] eductive fibre 3551 at junction point 3560. The electric signal is not propagated into non-conductive fibre 3551 at junction point 3560 because non-conductive fibre 3551 cannot conduct electricity. Junction point 3560 can refer to any point where adjacent conductive fibres and non-conductive fibres are contacting each other (e.g. touching). In the embodiment shown in FIG. 11, non-conductive fibre 3551 and conductive fibre 3502 are shown as being interlaced by being woven together. Weaving is only one exemplary embodiment of interlacing adjacent conductive and non-conductive fibres.

It should be noted that non-conductive fibres forming non-conductive network 3556 are also interlaced (e.g. by weaving, etc.). Non-conductive network 3556 can comprise non-conductive fibres (e.g. 3551 and 3564) and can also comprise conductive fibres that are not electrically connected to conductive fibres transmitting the electric signal.

The electric signal continues to be transmitted from junction point 3560 along conductive fibre 3502 until it reaches connection point 3561. Here, the electric signal propagates laterally (e.g. transverse) from conductive fibre 3552 into conductive fibre 3559 because conductive fibre 3559 can conduct electricity. Connection point 3561 can refer to any point where adjacent conductive fibres (e.g. 3552 and 3559) are contacting each other (e.g. touching). In the embodiment shown in FIG. 10, conductive fibre 3552 and conductive fibre 3559 are shown as being interlaced by being woven together. Again, weaving is only one exemplary embodiment of interlacing adjacent conductive fibres.

The electric signal continues to be transmitted from connection point 3561 along the electric pathway through a plurality of connection points 3561 to connector 3554. At least one conductive fibre of network 3555 is attached to connector 3554 to transmit the electric signal from the electric component 18 (e.g. network 3555) to connector 3554. Connector 3554 can be connected to a power source (not shown) to complete the electric circuit.

In the preceding description, for purposes of explanation, numerous details are set forth in order to provide a thorough understanding of the embodiments. However, it will be apparent to one skilled in the art that these specific details may not be required.

The above-described embodiments are intended to be examples only. Alterations, modifications and variations can be effected to the particular embodiments by those of skill in the art without departing from the scope, which is defined solely by the claims appended hereto.

We claim:

1. A method of forming a three-dimensional conductive patch, the three-dimensional conductive patch forming a base layer coupled to one or more loop sections extending transverse o the base layer, the method comprising:
forming the base fabric surface by interlacing a plurality of fibres including non-conductive fibres;
forming a first segment including conductive fibres as a first portion of the three-dimensional conductive patch by interlacing a plurality of the conductive fibres transverse to the base fabric surface, the first portion interlaced with a first base fibre of the base fabric surface at one end and in a direction to an apex distanced from the base surface layer at another end of the first segment; and
forming a second segment as a second portion of the three-dimensional conductive patch by interlacing a plurality of fibres including the conductive fibres extending in a direction from the apex to a second base fibre of the base surface layer;
wherein the second portion is positioned relative to the first portion via the first base fibre and the second base fibre such that the first and second portions form a loop extending from the base fabric surface, the loop having the apex spaced apart from the base fabric surface, the first portion, the second portion and the base fabric surface integral with each other.

2. The method of claim 1, wherein the forming of the second portion comprises connecting the second base fibre to the first base fibre.

3. The method of claim 1, wherein the apex has one or more fibres.

4. The method of claim 1, wherein at least one of the first portion and the second portion comprise one or more non-conductive fibres to facilitate maintaining of said extending of the loop.

5. The method of claim 1, wherein the first base fibre is coupled to a first non-conductive fibre and the second base fibre is coupled to a second non-conductive fibre, each of the first and second non-conductive fibres integral with the layer.

6. The method of claim 5, wherein at least one of the first non-conductive fibre and the second non-conductive fibre are coupled to an adjacent conductive fibre, the adjacent conductive fibre extending from the layer adjacent to the base fabric surface and adjacent to at least one of the first part and the second part of the loop to electrically connect the loop to an adjacent loop.

7. The method of claim 1, wherein the first base fibre is positioned in the first part of the loop and the second base fibre is positioned in the second part of the loop.

8. The method of claim 1, wherein the first base fibre and the second base fibre are positioned in the first portion of the layer such that the first base fibre and the second base fibre are adjacent to each other.

9. The method of claim 1, wherein the layer comprises a second loop extending from the base fabric surface and having a second apex spaced apart from the first portion, the second loop positioned between the first loop and the second base fibre.

10. The method of claim 9, wherein at least one of the first part and the second part comprise a non-conductive fibre to provide electrical insulation between the loop and the second loop.

11. A garment comprising the conductive patch according to claim 1.

12. The garment of claim 11, further comprising:
one or more electrical connectors attached to the layer, the one or more electrical connectors for facilitating receipt and transmission of electrical signals between a controller and the three-dimensional conductive patch when the controller is connected to the three-dimensional conductive patch; and
a conductive pathway consisting of one or more conductive fibres interlaced in the layer as part of the plurality of fibres, the conductive pathway electrically connected to the one or more electrical connectors and to the three-dimensional conductive patch.

13. The garment of claim 12, wherein the garment includes a first region in the layer containing the conductive patch and a second region in the layer adjacent to the first region, the first region having a lower degree of elasticity reflected by the plurality of fibres therein relative to a degree of elasticity reflected by the plurality of fibres in the second region.

14. The garment of claim 13, wherein the second region contains non-conductive fibres for electrically insulating the three-dimensional conductive patch from a second three-dimensional conductive patch in the layer.

15. The garment of claim 13, wherein a knit type of the plurality of fibres in the first region is different from a knit type of the plurality of fibres in the second region, such that said difference is a factor providing said first region having a lower degree of elasticity reflected by the plurality of fibres therein relative to the degree of elasticity reflected by the plurality of fibres in the second region.

16. The garment of claim 13, wherein the plurality of fibres in the first region includes both conductive fibres connected to the conductive pathway and non-conductive fibres.

17. The garment of claim 13, wherein the plurality of fibres in the first region have a higher knit density (threads per inch) than the plurality of fibres in the second region.

18. The garment of claim 13, wherein the plurality of fibres themselves in the first region have a lower degree of elasticity than the plurality of fibres in the second region.

19. The garment of claim 11, wherein the loop extends from the base fabric surface in a transverse direction to contact an underlying body portion of a wearer to inhibit movement of the garment adjacent to the underling body portion when worn by the wear.
